(19) United States
(12) Patent Application Publication
(10) Pub. No.: US 2010/0086530 A1
(43) Pub. Date: Apr. 8, 2010

(54) Cancer-tumor necrosis by artery nutrient-oxygen blocking

(76) Inventor: Norman P. Martinov, Woodland Hills, CA (US)

Correspondence Address: NORMAN P. MARTINOV, 4744 DEL MORENO DRIVE, WOODLAND HILLS, CA, 91364, US

(21) Appl. No.: 12/586,809

(22) Filed: Sep. 29, 2009

Related U.S. Application Data
(60) Provisional application No. 61/195,248, filed on Oct. 6, 2008.

(51) Int. Cl. A61K 35/14; A61P 35/04

(52) U.S. Cl. 424/93.73

(57) ABSTRACT

This is a new, novel, natural, relatively non-invasive and non-toxic method for the shrinkage and necrosis of cancerous cells, tumors, glands, organs, metastases processes or unwanted growths in human or animal patients. A patient's or other donor's blood is aged and processed which results in the loss of its blood vessel dilating component, nitric oxide. In addition to the loss of the vessel dilating component the other components of the blood become stiffer, stickier and sludgier. This processed blood is to then be directed by injection or catheter into the newly formed, fine, fragile, deranged and disorganized micro-capillaries of the patients cancerous tumors or unwanted growths or into the arteries that feed them nutrients and oxygen. The processed blood blocks, clogs or clots these super-fine micro-vessels and their wasteline pathways thereby causing the cancerous tumors or unwanted growths to shrink and die. The processed, aged or modified blood can also be used in combination with a blood separating hemapheresic means which can separate out the nitric oxide and/or any other components which would make the blocking blood more fluid or less viscous and can also be used immediately without further aging. In addition, the hemapheresic means can also separate other blocking components of the blood such as artery plaque building agents which together with the nitric oxide depleted blood will more readily block the unwanted growths micro-capillaries. Some or all of the unused components of the hemapheresic separated blood can be returned to the donor, harvested for future use or discarded. The method can also be used periodically to control or treat the condition if no cure is possible. Furthermore, the method can be used on many other chronic conditions which require new blood vessel formation to cause and/or sustain them. Finally, these methods can also be used in combination or conjunction with other therapies, procedures and protocols that seek the same end result for possible synergistic advantages.

### CROSS-REFERENCE TO RELATED APPLICATIONS

This application is entitled to the benefit of Provisional Patent Application Ser. No. 61/195,248 filed 2008 Oct. 6.

### STATEMENT REGARDING FEDERALLY SPONSORED RESEARCH DEVELOPMENT

Not Applicable.

### BACKGROUND

1. Field of Invention

This invention relates to developing a novel, natural, non-invasive cancer-tumor-unwanted growth or process necrosis therapy-method-protocol. This therapy would be a kinder, gentler method than the present conventional surgery, radiation or chemotherapy (cut, burn or poison) therapies. This proposed method can replace the conventional therapies or be used in addition to them (before, during or after) to minimize their debilitating effects or synergistically improve them. Most traditional cancer therapies and unwanted growth-process necrosis means are specific to the type of maladies and are not targeted. This method of cancer cell, tumor, unwanted growth or process necrosis has the potential to be targeted to a common process, which is the blocking/clogging of the newly formed, fine, deranged and disorganized nutrient supply and wasteline angiogenic vasculature of the cancercell-tumors or the metastases process.

2. Description of Prior Art

Most present day cancer therapies are not targeted to all cancers, tumors (benign or malignant), metastases processes or unwanted growths. Furthermore, most are usually toxic or invasive such as surgery, radiation or chemotherapy (cut, burn or poison) which can kill normal cells and/or compromise the patient's immune system. Also, some tumors are unresectable or need to be modified before surgery can be performed to remove them. Many conventional drug therapies cause contra-indications and can be highly toxic. Most present day therapies and protocols do not use natural bodily components to shrink or destroy cancerous tumors or unwanted growths and therefore require long clinical trial procedures to be approved.

### SUMMARY

This is a novel, natural, non-invasive and non-toxic therapy-method to shrink or destroy cancerous tumors or unwanted growths in humans or animals using autologous aged, processed or modified whole blood and/or components thereof derived from the patient. The whole blood is withdrawn from the patient whereupon it is aged and processed resulting in a stiffer, stickier, viscous form especially with the loss of the nitric oxide component of the blood. The aged and/or processed blood is then re-infused into the patient's cancerous tumor, gland, organ or unwanted growth by needle or catheter into the artery feeding nutrients and oxygen to the cancers or unwanted growth. The aged-modified blood should conspire to block, clog, coagulate and constrict the fine, fragile, deranged and disorganized micro-capillaries of the cancerous cells, tumors or unwanted growths and/or the metastases process thereby denying them the needed nutrients and wasteline drainage pathways resulting in tumor shrinkage and/or destruction. The patient's whole blood autologous donation can be used in combination with a hema-pheresis apparatus to separate the components of the blood, especially the nitric oxide, whereby separated components can be returned to the patient, harvested or discarded while the capillary blocking, clogging components can be directed to the patient's tumors or unwanted growths or the arteries which feed them or can be stored and further aged for future use. Another method to modify the blood to remove the vessel dilating component nitric oxide is to treat it with free hemoglobin before perfusion into the artery feeding the cancerous tumors, glands, organs or lymph nodes. Similarly, the methods above are to be used with allologous compatable blood, allologous incompatable blood, animal blood or synthetic blood and/or their components. In addition to shrinkage and necrosis of malignant tumors and disruption of the metastases process by the here-in-above method-protocols, another purpose is shrinkage and necrosis of benign tumors and the unwanted growths which depend upon micro-vessel angiogenisis. These unwanted growths can range from macular degeneration to rheumatoid arthritis and can include more than seventy-five diseases and conditions including uterine fibroids, endometriosis and arterial plaque build-up. In regard to the hema-pheresic feature here-in-above the targeted micro-capillaries can be blocked, clogged or embolized by separation of the arterial plaque build-up and/or coagulation components from the blood which are then directed to the said micro-capillaries with the balance of blood components returned to the patient, harvested or discarded. All the here-in-above methods and protocols can also be used in combination with (before, during or after) present or future conventional or alternative therapies, protocols or procedures including anti-angiogenic and other drug therapies.

### Objects and Advantages

Accordingly, several objects and advantages of the present invention are:

(a) to provide a relatively non-invasive, natural and non-toxic method-therapy-protocol to shrink or destroy cancerous cells, tumors (malignant or benign) of tissue, gland, organ or lymph nodes or unwanted growths

(b) to use a patient's own aged, processed, separated or modified blood and/or components to block or clog micro-vessels feeding nutrients or removing waste from the cancerous tumors or unwanted growths

(c) to use other compatable donated aged blood and/or components as blocking agents

(d) to use other incompatable donated aged blood and/or components as blocking agents

(e) to use animal donated blood and/or components as blocking agents

(f) to use synthetic blood and/or components as blocking agents

(g) to combine blood separation means with blood donation means especially the autologous donation process

(h) to separate and remove the blood vessel dilating Nitric Oxide component from the blood before perfusion of the blood to the tumor or unwanted growths or arteries feeding them

(i) to remove the blood vessel dilating Nitric Oxide component by treating it with hemoglobin before perfusion of the blood to the tumor or unwanted growths or arteries feeding them

(j) to provide convenient and cost-effective treatment by using the autologous method where donor and patient are one

(k) to provide non-infectious and non-immune reaction treatment by using the autologous method where donor and patient are one

(l) to use the method as a chronic treatment to control and maintain the cancerous tumor, gland, organ, lymph nodes or unwanted growths including the many chronic conditions which rely on newly formed, fine, fragile, deranged and disorganized angiogenesis required to initiate, sustain and promote them such as, but not limited to inflammation, auto-immune diseases, atherosclerosis, arterial plaque, obesity fat cells, kaposi's sarcoma, hemangiomas, neurofibromatosis psoriasis, leprosy, varicose veins, macular degeneration, diabetic retenopathy, pterygeum, endometriosis, uterine fibroids, benign prostatic hyperplasia, rhematoid and other arthritis's, etc.

(m) to use the hemapheresic separation of atherosclerotic arterial plaque build-up components of the here-in-above mentioned blood sources directed to the cancerous tumors, glands, organs, lymph nodes, unwanted growths or other chronic conditions fine, fragile, disorganized, deranged and leaky micro-capillaries or to the arteries feeding them so as to block, clog or clot them thereby denying them nutrients and oxygen and waste pathways causing their shrinkage and/or necrosis while simultaneously improving the donor's circulatory perfusion system

(n) to use the micro-vessel blocking methods in combination with conventional and alternative cancer, etc. curing therapies and/or protocols (before, during or after) including anti-angiogenesis expression drugs for possible synergistic effect

Further objects and advantages of my invention will become apparent from a consideration of the ensuing conclusions, ramifications, scope and detailed description.

### Conclusion, Ramifications and Scope

Other ramifications, scope and enlightenment can be found in the Provisional Patent Application referenced in this application.

It should be observed that the main basis for blocking or clogging the micro-capillaries of the unwanted cancer, growths or processes is the use of aged blood (autologous, allologous or synthetic) and/or their components. It has been documented that aged, processed blood used for transfusion in heart surgery patients has fared badly due to its increased stickiness, stiffness and loss of the vessel dilation component, nitric oxide. It is theorized that the red blood cells and/or other components of the aged blood can block or clog the angiogenesis of the newly formed fragile micro-vessels of unwanted growths especially solid tumors and the metastases process at the end of their arterial supply line and since this therapy can attack the common process it should be effective on all tumors. Furthermore, since this therapy-protocol blocks newly-formed disorganized, fragile micro-capillaries while largely avoiding the established vessels, it should be considered a targeted therapy.

In regard to the hempheresic separation and use of arterial plaque build-up components such as, LDL cholesterol, triglycerides, VLDL, platelets, fibrinogin, etc., to block or clot the the tumors micro-vasculature, with the balance of the components returned to the donor, harvested or discarded. it should be observed that this will also improve the donors circulatory system against heart attack and/or stroke.

The use of these new methods in combination or conjunction with present or future conventional or alternative therapy-protocols should improve their efficacy and lessen the dibilatating effects of the more invasive ones. In regard to radiation and chemotherapy, these new therapies can be used in a metronomic fashion.

These new method-therapies may lend themselves to being periodical chronic treatments used to control tumors and/or unwanted growths in which case the autologous donor method would be the most practical especially in regard to convenience, cost, non-infection and non-immune rejection. Note that the autologous method would be favored in all cases including the hemapheresic plaque build-up separation method mentioned here-in-above for all the same reasons.

Since this invention is a method-process and not a mechanical device, this application-specification does not contain drawings.

### DETAILED DESCRIPTION OF THE INVENTION

This is a new method of using aged, separated or modified whole human or animal blood and/or components thereof for a new purpose of blocking, clogging or clotting the newly formed, fine, fragile, deranged and disorganized micro-vasculature and wasteline pathways of cancerous cells, tumors or unwanted growths thereby denying them nutrients and/or oxygen or waste pathways and causing their shrinkage and/or necrosis. The aged human or animal whole blood and/or components thereof can be obtained autologously or allologously. The allologous blood can be compatable or incompatable with the human or animal patient. Another source of micro-vessel blocking blood can be a synthetic formulation. The treated, separated or modified blood and/or components are to be injected, or delivered by catheter to the cancerous cells, tumor, gland, organ, lymph node or unwanted growth or into an artery feeding them. The aged blood and/or components lose their fluidity and become stiffer, stickier and sludgier and combined with the loss of the blood vessel dilating component nitric oxide conspire to block, clog or clot the newly formed, fine, fragile micro-capillaries of tumors or the metastases process. If the method is to be used more than one time or as a chronic treatment for control of the diseased condition a catheter can be left in place until the treatment is finished. In conjunction with this method of treatment is a hemapheresic apparatus to separate the more active blocking/clogging components which are to be directed to the patient's tumors or unwanted growths or arteries feeding them while the remaining components are returned to to the patient, harvested or discarded. The hemaphersic apparatus can also be used to remove the nitric oxide vessel dilating component so that the vessel blocking blood and/or its components can be used immediately instead of relying on the aging process for its removal. Another method to remove nitric oxide from whole blood is to treat it with hemoglobin, again for possible immediate use as a micro-vessel blocker. The methods above using separated artery plaque build-up components such as LDL (low density lipoids), VLDL (very low density lipoids), triglicerides, platelets, fibrinogin, etc. and directing them to the patient's tumors or unwanted growths or arteries feeding them while the remaining components are returned to the patient, harvested or discarded. Use of any or all of the here-in-above mentioned methods in combination or in conjunction (before, during, after or metronomically) with present day or future conventional or alternative cancer curing therapies or protocols including anti-angiogenesis drug therapies. Use of any or all of the here-in-above methods to block and clog the nutrient oxygen supply and/or waste removal of ailments, conditions or diseases that rely on micro-capillary angiogenesis to cause and/or sustain them including, but not limited to, rheumatoid and other arthritis, macular degeneration and other eye conditions, uterine fibroids and endometriosis, benign prostatic hyperplasia (BPH), cysts, leprosy, neurofibromatosis, hemangiomas and arterial plaque build-up.

I claim:

1. A method of using aged, separated and/or modified human or animal autologous whole blood and/or components thereof for a new purpose of blocking, clogging or clotting the fine, fragile, deranged and disorganized micro-capillaries of cancerous cells, tumors, glands, organs, lymph nodes, unwanted growths or the metastases process by injection and/or catheter means into these unwanted growths or the arteries feeding them, thereby blocking their nutrient and oxygen supplies and wasteline pathways causing their shrinkage and/or necrosis.

2. The method of claim 1 in combination with a hemapheresic apparatus means to separate blocking components of the blood and direct them to the micro-vessels of the unwanted growths or arteries feeding them and with some or all of the remaining components returned to the patient's systemic blood supply, harvested or discarded.

3. The method of claim 2 whereby the vessel dilating component, nitric oxide, is separated from the blood, harvested or discarded and with some or all of the blocking components directed to the micro-vessels of the unwanted growths or arteries feeding them.

4. The method of claim 2 whereby the arterial plaque building clotting components of the blood are separated and directed to the micro-vessels of the unwanted growths or arteries feeding them and with some or all of the remaining components returned to the patient, harvested or discarded.

5. The method of claim 1 in combination with hemoglobin treatment of the blood to remove the nitric oxide and harvest or discard it and with some or all of the remaining blocking components directed to the micro-vessels of the unwanted growths or arteries feeding them.

6. The method of claim 1 whereby the human or animal whole blood used is allologous and compatable with the patient.

7. The method of claim 1 whereby the human or animal whole blood used is allologous and incompatable with the patient.

8. The method of claim 1 whereby the whole blood used is a synthetic blood formulation.

9. The method of claim 1 in combination (before, during, after or metronomically) with present day or future cancer or unwanted growths cures, methods, therapies, procedures or protocols.
